(19) United States
(12) Patent Application Publication
(10) Pub. No.: US 2013/0115266 A1
(43) Pub. Date: May 9, 2013

(54) EDIBLE WAFER-TYPE PRODUCT FOR DELIVERY OF NUTRACEUTICALS AND PHARMACEUTICALS

(71) Applicant: BestSweet, Inc., Mooresville, NC (US)

(72) Inventors: Richard C. Zulman, Cornelius, NC (US); Anthony J. Bell, Andover, NJ (US); Mark Brian Jones, Cleveland, NC (US)

(73) Assignee: BestSweet, Inc., Mooresville, NC (US)

(21) Appl. No.: 13/666,182

(22) Filed: Nov. 1, 2012

Related U.S. Application Data
(60) Provisional application No. 61/555,589, filed on Nov. 4, 2011.

(51) Int. Cl. A61K 9/00; B29C 70/00; B29C 37/00; A23L 1/30; A23P 1/08

(52) U.S. Cl. 424/441; 426/94; 426/282; 426/284; 264/138; 264/250

(57) ABSTRACT

A food product for carrying and delivery nutraceutical or pharmaceutical or both is provided. The food product may be an edible wafer-based outer layer and an inner layer of an edible filling comprising the nutraceutical or pharmaceutical or both.

### RELATED APPLICATIONS

This application claims priority to U.S. Provisional Patent Application Ser. No. 61/555,589, filed Nov. 4, 2011 the contents of which is hereby incorporated by reference in its entirety.

### FIELD OF THE INVENTION

The present invention generally relates to an edible wafer-type food product for delivery of various health-related supplements.

### BACKGROUND OF THE INVENTION

The consumption of various supplements to improve health has seen a tremendous growth in popularity. Health supplements including nutraceuticals, such as vitamins, minerals, or herbal remedies, and pharmaceuticals are being taken in vast quantities by consumers and are being recommended by a myriad of health care providers. Often such supplements are in powder or capsule form. Many consumers, however, do not like the taste or texture of the supplement. An alternative are pills or tablet which have no taste. Pills and tablets, however, are limited by many consumers not liking to swallow pills or tablets.

Thus, better tasting means for delivering supplements are desired. For example, U.S. Patent Publication 2003/0017202 to Bunick et al. describes a coated soft core consumable product with an active that can include calcium carbonate. If the active has an objectionable taste, it is coated with a taste masking agent. U.S. Publication No. 2009/0011079 to Overly provides a hard coated confectionary product having a fortified soft chewing core. Such products, however, still often have taste or texture issues. Thus, there remains a need for a means for delivering supplements that is perceived as tasteful and easy to consume.

### SUMMARY OF THE INVENTION

To this end, the present invention provides a wafer-type food product for carrying and delivering a nutraceutical or pharmaceutical. The food product comprises an edible wafer outer layer and an inner edible filling layer comprising the nutraceutical or pharmaceutical. The edible wafer outer layer can be in the form of a hollow tube or pillow with the filling inside or can be in the form of a sandwich with the filling between two or more wafer layers. The present invention also provides a method for producing such a food product.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 provides a flow chart that demonstrates an exemplary process of preparing a food product according to the present invention.

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

The term “consists essentially of” (and grammatical variants), as applied to the methods in this invention, means the methods or compositions can contain additional steps as long as the additional steps or components do not materially alter the basic and novel characteristic(s) of the present invention.

The term “consisting of” excludes any additional step that is not specified in the claim.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

All patents, patent applications and publications referred to herein are incorporated by reference in their entirety. In case of a conflict in terminology, the present specification is controlling.

As one of ordinary skill in the art may appreciate, the parameters described herein may vary greatly depending on the process, formulation and/or apparatus as well as the desired properties of the final product.

One aspect of the invention provides a food product for carrying and delivering a nutraceutical or pharmaceutical or both. The food product may be an edible wafer-based outer layer and an inner layer of an edible filling comprising the nutraceutical or pharmaceutical or both. The edible wafer outer layer can be in the form of a hollow tube or pillow with the filling inside or can be in the form of a sandwich with the filling between two or more wafer layers.

The edible wafer outer layer may be flour-based and may be baked. Suitable dry ingredients may include flour, including, but not limited to, whole wheat, brown rice, gluten free, unbleached unbromated all purpose, oat, and/or soy flour; fiber sources, including, but not limited to, guar gum, acacia, oat bran, corn bran, psyllium husks, almond meal, and/or flax seeds, leavening agents, including, but not limited to, sodium bicarbonate and/or baking powder, flavoring agents, natural and artificial, including, but not limited to, cocoa powder, cinnamon, spices, extracts, and oils, including resveratrol, and certain natural and artificial sugars such as turbinado, evaporated cane juice, fructose, date sugar, brown sugar, stevia, aspartame, saccharin, acesulfame potassium and/or cyclamates.

Suitable wet ingredients may include egg whites, sugars, including, but not limited to, unsulfured molasses, and/or brown-rice syrup, and flavoring agents, including, but not limited to, pure bean and nut extracts and/or oils, nut butter, and/or pureed fruit.

The filling is non-aqueous, i.e., has a no free or tied up moisture, e.g., has a moisture content of less than 2 percent, and may include sugars, including, but not limited to, confectioner sugar, turbinado, evaporated cane sugar, fructose, unsulfured molasses, date sugar, brown rice syrup, brown sugar, and/or stevia, salt, gelling agents such as pectin, gum, or gelatin, fats such as butter, oils, sugar substitutes such as isomalt, and flavoring agents.

As used herein “nutraceutical” supplement include any nutrients that may provides health and medical benefits, including the prevention and treatment of disease. Examples include, but are not limited to, vitamins, minerals, probiotics, enzymes, herb and other botanical extracts, amino acid, concentrates, metabolites, constituents, etc.

Exemplary vitamins and minerals include, but are not limited to, vitamins A (in the form of, for example, palmitate or beta carotene), B-complex (such as B-1, B-2, B-6 and B-12), C, D, E and K; niacin; acid vitamins such as pantothenic acid and folic acid; biotin; minerals such as iron, calcium, magnesium, iodine, copper, phosphorus, zinc, manganese, potassium, chromium, cobalt, molybdenum, selenium, nickel, tin, silicon, vanadium and boron; nutraceutical supplements such as fluorine and chlorine; and the like. In one embodiment, vitamins and minerals are included in such levels as to provide a significant dietary supplement, such as according to the U.S. Recommended Daily Allowance (US RDA). For example, in some embodiments of the present invention, it is suggested that the filling contain 100% or more of the US RDA (such as the U.S. RDA established by the U.S. Food and Drug Administration and revised in 1980) of some or all of the vitamins and minerals per dose of food product consumed. Various herbs and herbal remedies can be utilized as the nutraceutical supplements. The herbs are generally selected from those which have various medicinal or dietary supplement properties. Herbs are generally aromatic plants or plant parts that can be used medicinally or for flavoring. Examples include Gingko biloba, gotu kola, echinacea, St. John's wort, ginseng, valerian and the like. Suitable herbs can be used alone or in various mixtures in the filling described herein.

As used herein “pharmaceutical” is defined as any chemical substance intended for use in the medical diagnosis, cure, treatment, or prevention of disease, for example over-the-counter drugs (OTC) and prescription only medicine (POM). Exemplary active pharmaceutical components are listed in U.S. Pat. No. 6,723,358, column 9, line 25 to column 13, line 25, the disclosure of which is incorporated herein by reference in its entirety, and may include NSAIDS, cholesterol-reducing drugs, and probiotics.

Optionally, the nutraceutical and/or the pharmaceutical may be included in the batter.

In general, the supplement of the filling or the filling and batter, may be formulated alone or in combination to promote various specific physiological effects including sexual potency, endurance, cardiovascular health, control fat and cholesterol levels, healthy joints, bone density levels, appetite levels, antioxidant levels and the like.

The food product may be in the shape of a hollow cream-filled tube, a cream-filled pillow, or a sandwich with at least one layer of filling sandwiched between at least two layers of the edible wafer.

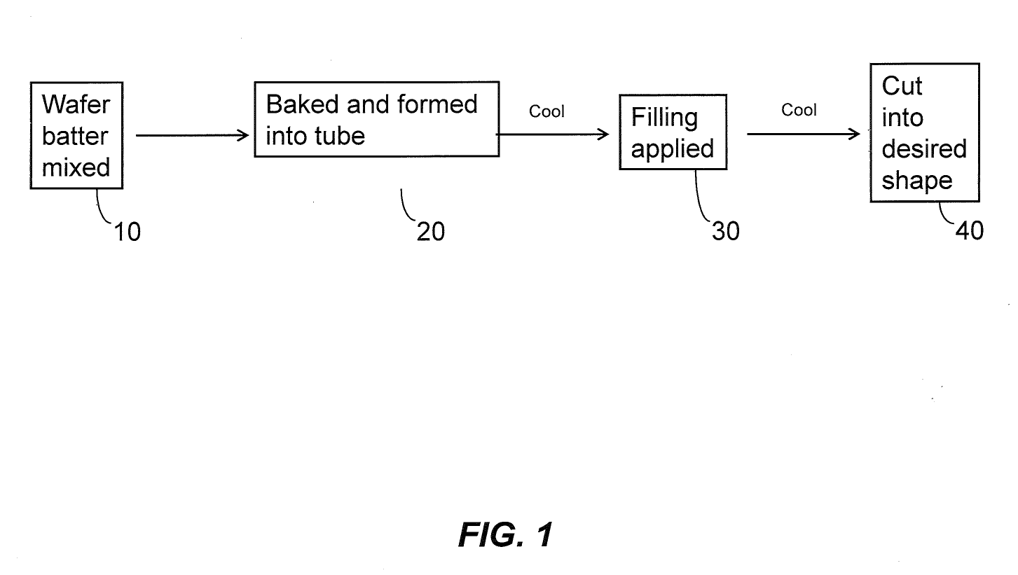

Referring to FIG. 1, an exemplary method for making the food product is provided. The wafer batter is mixed 10. The batter is formed into the desired predetermined shape and baked 20. The baked shape is cooled. The filling is then applied 30 (e.g., injected) into the hollow tube. The filled shape is allowed to cool. The filled shape is then cut 40 into the desired end shape.

The following examples further illustrate the invention.

### EXAMPLES

### Example 1

Wafer

| Ingredient | Wet amount | Wet % |
|---|---|---|
| Water | 4.8354 | 54.550 |
| Flour Salt | 0.0151 | 0.170 |
| Sodium Bicarbonate | 0.0115 | 0.130 |
| Granulated Sugar | 0.1773 | 2.000 |
| Corn Starch | 0.1330 | 1.500 |
| Wheat Flour | 3.6361 | 41.020 |
| Soy Lecithin | 0.0186 | 0.210 |
| Coconut Oil | 0.0372 | 0.420 |
| | 8.8642 | 100.000% |

Starting Solids = 45.01%
Cooked Solids = 99.75%

Filling

| Ingredient | Wet amount | Wet % |
|---|---|---|
| Plant Stanol | 2.8880 | 72.200 |
| Lecithin | 0.0445 | 1.113 |
| 6× Sugar | 0.6275 | 15.687 |
| Cocoa Powder | 0.3200 | 8.000 |
| Milk Powder | 0.1200 | 3.000 |
| | 4.0000 | 100.000% |

Total Solids = 99.449%

### Example 2

Wafer

| Ingredient | Wet amount | Wet % |
|---|---|---|
| Water | 4.8354 | 54.550 |
| Flour Salt | 0.0151 | 0.170 |
| Sodium Bicarbonate | 0.0115 | 0.130 |
| Granulated Sugar | 0.1773 | 2.000 |
| Corn Starch | 0.1330 | 1.500 |
| Wheat Flour | 3.6361 | 41.020 |
| Soy Lecithin | 0.0186 | 0.210 |
| Coconut Oil | 0.0372 | 0.420 |
| | 8.8642 | 100.000% |

Starting Solids = 45.01%
Cooked Solids = 9.75%

Filling

| Ingredient | Wet amount | Wet % |
|---|---|---|
| Plant Stanol | 2.8880 | 72.200 |
| Lecithin | 0.0445 | 1.113 |
| 6× Sugar | 0.6275 | 15.687 |
| Cocoa Powder | 0.2800 | 7.000 |
| Aspirin Powder | 0.0820 | 2.050 |
| Milk Powder | 0.0780 | 1.950 |

Total Solids = 99.528%

Having thus described certain embodiments of the present invention, it is to be understood that the invention defined by the appended claims is not to be limited by particular details set forth in the above description as many apparent variations thereof are possible without departing from the spirit or scope thereof as hereinafter claimed.

What is claimed:

1. A food product for carrying and delivering a nutraceutical or pharmaceutical or both comprising an edible wafer outer layer and an inner edible non-aqueous filling layer comprising a nutraceutical or pharmaceutical or both.

2. The food product according to claim 1, wherein the outer layer is a hollow molded-shaped tube.

3. The food product according to claim 1, wherein the outer layer is in the form of a sandwich comprising two or more layers of edible wafer.

4. The food product according to claim 1, wherein the edible wafer outer layer includes the nutraceutical or pharmaceutical or both.

5. A method of forming a food product for carrying and delivering a nutraceutical or pharmaceutical or both comprising forming an edible wafer outer layer in a predetermined shape, filling the edible wafer layer with an edible filling comprising a nutraceutical or pharmaceutical or both, and optionally cutting the filled wafer edible outer layer into an end shape of the food product.

6. The method according to claim 5, wherein the end shape is a hollow molded-shaped tube.

7. The method according to claim 5, wherein the edible wafer outer layer includes the nutraceutical or pharmaceutical or both.
